(19) United States
(12) Patent Application Publication
(10) Pub. No.: US 2025/0239373 A1
(43) Pub. Date: Jul. 24, 2025

(54) INCORPORATING POPULATION-LEVEL KNOWLEDGE INTO CONDITIONAL AVERAGE TREATMENT EFFECT ESTIMATION

(71) Applicant: Microsoft Technology Licensing, LLC, Redmond, WA (US)

(72) Inventors: Niranjani PRASAD, Cambridge (GB); Javier GONZALEZ HERNANDEZ, Cambridge (GB); Aditya Vithal NORI, Cambridge (GB)

(21) Appl. No.: 18/421,799

(22) Filed: Jan. 24, 2024

(51) Int. Cl. G16H 50/70

(52) U.S. Cl. CPC G16H 50/70

(57) ABSTRACT

Example solutions incorporate population-level information into an estimation procedure of a conditional average treatment effect (CATE) by: receiving observational data associated with a medical treatment; receiving average treatment effect (ATE) data associated with the medical treatment performed across a population of individuals; training a model using at least the observational data and the ATE data, the model being trained to generate at least a conditional average treatment effect (CATE) estimation for the medical treatment; applying patient data of a first patient as input to the model, thereby generating a CATE estimation indicating how the medical treatment would affect the first patient; and causing treatment to be applied to the first patient based on the CATE estimation.

### BACKGROUND

The Conditional Average Treatment Effect (CATE) is a concept used in healthcare and other fields to understand the effectiveness of a treatment for specific subgroups within a population. Unlike the Average Treatment Effect (ATE), which measures the average effect of a treatment across all individuals, CATE focuses on how the treatment effect varies across different subgroups defined by certain characteristics or conditions. In healthcare, this is particularly important because it acknowledges that patients may respond differently to a treatment based on various factors like age, gender, genetic makeup, or the presence of other health conditions.

### SUMMARY

The disclosed examples are described in detail below with reference to the accompanying drawing figures listed below. The following summary is provided to illustrate some examples disclosed herein. The following is not meant, however, to limit all examples to any particular configuration or sequence of operations.

Example solutions for providing a framework based on Gaussian process (GP) that incorporates population-level information into an estimation procedure of a conditional average treatment effect (CATE) include: receiving observational data associated with a medical treatment; receiving average treatment effect (ATE) data associated with the medical treatment performed across a population of individuals; training a GP model using at least the observational data and the ATE data, the GP model being trained to generate at least a conditional average treatment effect (CATE) estimation for the medical treatment; applying patient data of a first patient as input to the GP model, thereby generating a first CATE estimation identifying an estimation of how the medical treatment would affect the first patient; and causing the first CATE estimation to be displayed.

### BRIEF DESCRIPTION OF THE DRAWINGS

The disclosed examples are described in detail below with reference to the accompanying drawing figures listed below:

FIG. 1 illustrates an example precision health (PH) system that provides a GP-based framework that incorporates population-level information into an estimation procedure of a conditional average treatment effect (CATE);

FIG. 2 is a high-level description of a causal mechanism;

FIG. 3 is a high-level description of a causal mechanism;

FIG. 4 is a graph plotting example observational data that may be used to train the GP model of FIG. 1;

FIG. 5A and FIG. 5B illustrate differences between an example naïve approach to training a GP for CATE estimations versus the example GP training as described herein;

FIG. 6 is a flowchart illustrating exemplary operations that may be performed by the PH system of FIG. 1 for providing a GP-based framework that incorporates population-level information into an estimation procedure of a conditional average treatment effect (CATE); and

FIG. 7 is a block diagram of an example computing device (e.g., a computer storage device) for implementing aspects disclosed herein.

Corresponding reference characters indicate corresponding parts throughout the drawings. Any of the drawings may be combined into a single example or embodiment.

### DETAILED DESCRIPTION

The modelling of individual treatment effects has gained significant attention in the machine learning community. As large sources of observational data become more accessible, there is a natural shift from defining policies at the population level to specifying individual-level interventions. Machine learning plays a crucial role in this transition, particularly in the emerging field of precision health.

There have been numerous approaches developed for estimating the conditional average treatment effect (CATE), the key quantity when learning individual-level causal mechanisms. Estimating CATE is challenging as it models counterfactual outcomes that are never observed. Existing solutions for CATE estimation primarily rely on parametric models, regularized models, or machine learning-based techniques such as propensity score matching, double machine learning, and targeted maximum likelihood estimation. These methods, however, have largely overlooked how to incorporate, into the estimation process, population-level knowledge that may be available. Such data can be relevant in precision health, where patient-level inference about the effect of a treatment is carried out with post-market data in the presence of already publicly available results from previous randomized trials. Further, these methods often involve extrapolating beyond areas with data, considering the counterfactual's location, or using complex learning algorithms to estimate the individual treatment effects.

In contrast, a precision health system designs medical interventions tailored to individuals rather than targeting population-level effects. In this system, machine learning's primary role is to use observational data from ordinary medical practice to infer mechanisms for the CATE, enabling consistent and personalized decision support.

More specifically, the precision health system implements a principled probabilistic framework that incorporates into the estimation procedure of the CATE available population-level information given in the form of average treatment effects (ATE) or other relevant statistics. The statistics are from real-world evidence studies (RWE), where a personalized model is built on observational data, but population treatment effects are available from previous randomized control trials. In examples, the system implements Gaussian processes (GPs) to constrain CATE estimation in the presence of an oracle of the population ATE. This example framework is valid for any estimand of the CATE and can be easily generalized to other models. GP modelling provides at least two benefits: they naturally enable uncertainty quantification, and the final constrained CATE can be computed in closed form by using some ideas from the Bayesian quadrature and kernel mean embedding literature. This framework leverages prior knowledge of the average population effects to constrain the potential outcomes model, thereby enhancing CATE estimation. The framework also utilizes population characteristics to constrain the model rather than extrapolating them to individuals.

While described with reference to GP-based models, aspects of the disclosure are operable with any model or neural network that characterizes the functions described herein.

The various examples are described in detail with reference to the accompanying drawings. Wherever preferable, the same reference number is used throughout the drawings to refer to the same or like parts. References made throughout this disclosure relating to specific examples and implementations are provided solely for illustrative purposes but, unless indicated to the contrary, are not meant to limit all examples.

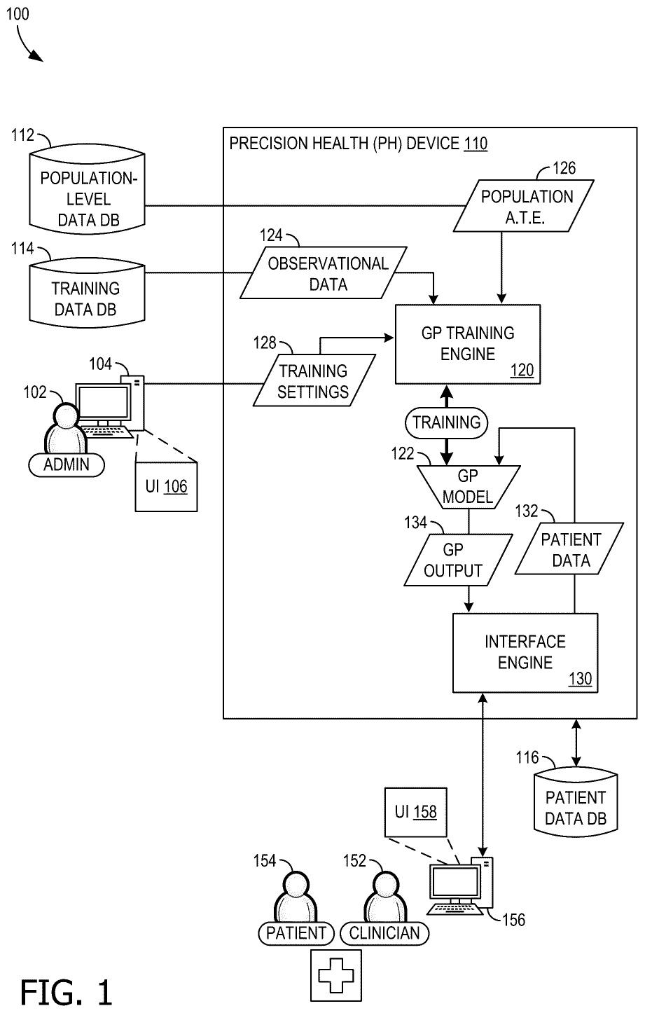

FIG. 1 illustrates an example precision health (PH) system 100 that provides a GP-based framework that incorporates population-level information into an estimation procedure of a CATE. In examples, a PH device 110 is a computing device that includes a GP training engine 120 and an interface engine 130. The GP training engine 120 is configured to train a GP model 122 that can be used to provide CATE estimations of a particular medical treatment (e.g., quantifying the effect of a particular drug) for a particular individual or group of individuals (e.g., individuals sharing some particular set of characteristics). Once the GP model 122 is trained, the interface engine 130 provides an interface through which an end user, such as a clinician 152, can view and consider treatment estimations for a particular individual, such as a patient 154, and for that particular treatment.

During a model training phase, an administrator (or “admin”) 102 configures the training of a particular GP model (e.g., the GP model 122) via an administrative computing device 104 (e.g., via application programming interface (API), or the like, provided by the PH device 110). The admin 102 identifies training settings 128 for the GP model 122, such as identifying what observational data 124 and population ATE 126 to use for the training. The observational data 124 may be sourced from a training data DB 114, and the population ATE 126 may be sourced from a population-level data DB 112 (e.g., a source of real-world evidence (RWE) studies).

Upon initiation of the training, the PH device 110 trains the GP model 122. More specifically, the GP training engine 120 uses the observational data 124 to train the GP model 122. Further, the GP training engine 120 also incorporates, into the estimation procedure of the CATE, available population-level information given in the form of average treatment effects (ATE, e.g., population A.T.E. 126) or other relevant statistics (e.g., from RWE studies or the like).

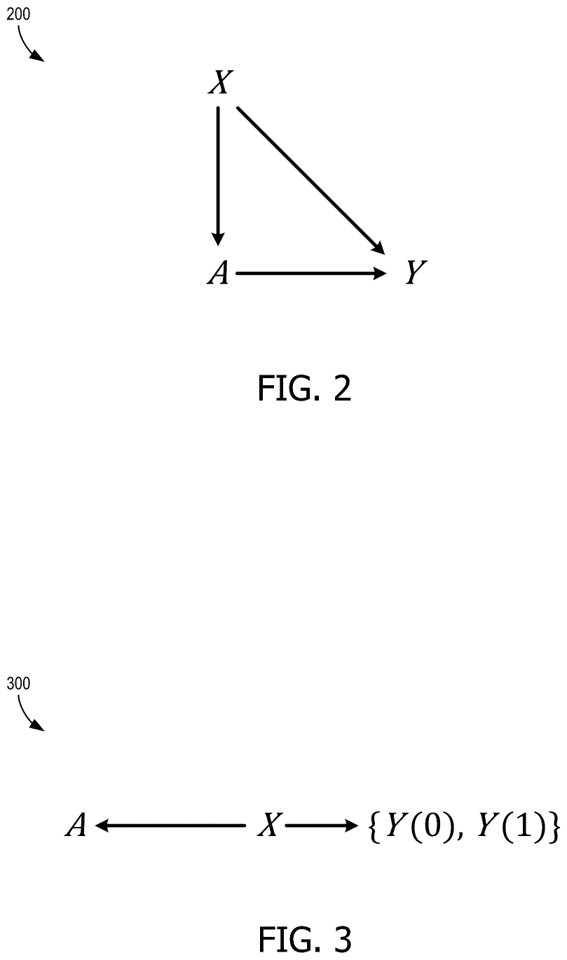

In examples, the model training phase starts the training of the GP model (or just “GP”) 122 with the observational data 124, namely a dataset [symbol] ={(a₁, y₁, X₁)}ᵢ₌₁ⁿ, where a₁={0,1} represents the absence or presence of a treatment of interest (control and case, respectively), y₁ represents a measure of the response of the patients to the treatment, and X₁ is a vector of metadata accounting for covariates like gender, age, treatment history, and the like. In matrix form, [symbol] ={a, y, X}. FIG. 2 is a high-level description of a causal mechanism 200. It is assumed that this sample has been generated from a joint probability measure [symbol] _{A,T,X} consistent with the casual mechanism shown in FIG. 2. As such, the multivariate random vector X accounts for all confounders between the treatment variable A and the response variable Y. Note that this assumption may be relaxed later.

The quantities of interest are expressed in terms of the potential outcomes formalism. For each observed individual, i, Y₁(0) and Y₁(1) (short notation for Y₁(A=0) and Y₁(A=1)) represent the potential outcome in the presence or absence, respectively, of the drug of interest, A={0, 1}. In the dataset [symbol] , only one of these two quantities is observed. The individual level causal effect is defined as:

$${τ}_{i} := {Y}_{i} ( 1 ) - {Y}_{i} ( 0 ) .$$

In the example PH system 100, individual effects beyond the observed sample are of interest. For this scenario, the conditional average treatment effect (CATE) is defined by:

$$τ ( X = x ) := [ Y ( 1 ) - Y ( 0 ) \vert X = x ]$$

The CATE accounts for the differences in the outcomes once the individual characteristics are set to X=x. The CATE allows the estimation of the effect of a treatment in a specific individual that has not necessarily been observed before (e.g., the patient 154).

Another quantity of interest is the average treatment effect (ATE). The ATE is the expectation of the CATE over the population of individuals of interest:

$$τ := {X}_{} [ Y ( 1 ) - Y ( 0 ) \vert X ] = {X}_{} [ {Y}_{k} ( 1 ) \vert X ] - {X}_{} [ {Y}_{k} ( 0 ) \vert X ] .$$

Under the assumptions in FIG. 2, the causal effects are identifiable and can be expressed in terms of functionals of the observed data. In particular:

$$τ = {X}_{} [ Y \vert X , A = 1 ] - {X}_{} [ Y \vert X , A = 0 ] .$$

The goal is to use the dataset [symbol] to provide an estimator \hat{τ}(x) of τ(x) under the causal assumptions made explicit in FIG. 2. Two extra factors are also introduced that have high relevance in practical applications (and that are usually ignored in known systems). The first factor is an assumption that, for the population of interest, [symbol] _{X}, the value of τ is known (e.g., from population ATE 126). In this sense, a population-level quantity is used to inform the estimation of an individual-level effect. This allows a smoothing of the transition between population-level inference and patient-level inference. The second factor is to quantify uncertainty and learn a full probability distribution over τ(x) rather than a point estimated. This is significant to make informed decisions about the class of patients for which the \hat{τ}(x) can reliably be used for decision making.

Considering potential outcomes and CATE estimation via generative modelling, one significant issue in causal inference is the observance of only one of the potential outcomes for each individual, i (e.g., either Y₁(0) or Y₁(1)). As such, the differences between factuals and counterfactuals are not accessible. Vectors denoted by y(1) and y(0) represent the response for a sample of n individuals assigned to the cases and controls, respectively. As such:

$$y := a \cdot y ( 1 ) + ( 1 - a ) \cdot y ( 0 ) ,$$

where a represents the vector of random vector of assignments. The counterfactual observations can be expressed as:

$${y}^{*} := ( 1 - a ) \cdot y ( 1 ) + a \cdot y ( 0 ) .$$

A model able to predict counterfactuals y* in an unbiased manner is used to estimate the CATE. Given the previous assumptions, the joint probability of the variables of the problem can be written in terms of a latent parameter vector, Θ, as:

$$ℙ ( y ( 0 ) , y ( 1 ) , a , X ) = \int {\prod }_{i = 1}^{n} ℙ ( {y}_{i} ( 0 ) , {y}_{i} ( 1 ) , a , X ; θ ) ℙ ( Θ ) d Θ .$$

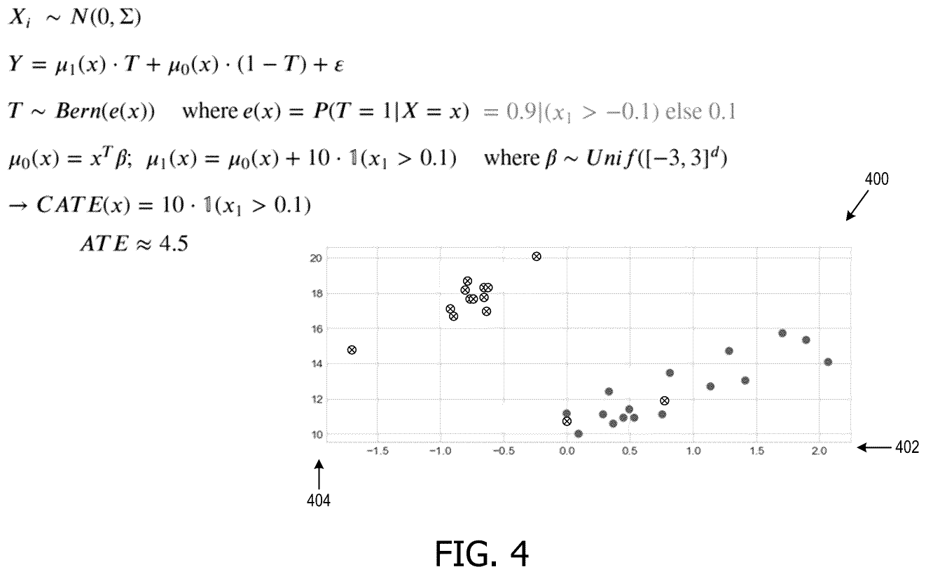

Assuming that the vector of parameters, Θ, is separable, the following factorizations of the joint are:

$$ℙ ( y ( 0 ) , y ( 1 ) , a , X \vert Θ ) = ℙ ( X ; {Θ}_{X} ) ℙ ( a \vert X , {Θ}_{A} ) ℙ ( y ( 0 ) , y ( 1 ) \vert X ; {Θ}_{Y} ) ,$$

where [symbol] (X; Θ_{X}) is environment, [symbol] (a|X, Θ_{A}) is assignment mechanism, and [symbol] (y(0), y(1)|X; Θ_{Y}) is science. FIG. 3 is a high-level description of a causal mechanism 300. In this example, the causal mechanism 300 is assumed here, using as output the potential outcomes Y(0) and Y(1) of the response. FIG. 3, which encodes in terms of the potential outcomes the same assumptions of FIG. 2, makes the above factorization explicit.

The above factorization includes three main components. The first component is the background characteristics of the units (population of interest), modelled by [symbol] (X; Θ_{X}). It is assumed that X is fixed, although there is some interest in modeling it because the observed matrix X are predictions of NLP models and therefore may be corrupted. The second component is the mechanism of assignment, given by [symbol] (a|X, Θ_{A}), that accounts for how the characteristics of an individual affects their probability of being assigned to the case or control group (0<P(a|X, Θ_{A})<1). The third component is the so-called model of the ‘science,’ [symbol] (y(0), y(1)|X; Θ_{Y}), that characterizes the probability of the response of a given individual for each potential outcome (e.g., response of an individual in the presence or absence of the drug). Note that this factorization is possible based on the assumptions of FIG. 3. If those assumptions do not hold, then there is no guarantee that the quantities derived here are causal. If they hold, however, it is possible to prove that the posterior over the counterfactuals only depends on the ‘model of the science’:

[symbol]

(y*|y,X,a,Θ_{Y})α [symbol] (y(0),y(1)|X;Θ_{Y}).

[symbol]

(y(0),y(1)|X; Θ_{Y}) is therefore the right object that needs to be modeled to have access to some estimates of the counterfactuals, the potential outcomes and of τ(x).

Regarding the probabilistic model of the ‘science’ with population-level information, aspects of the disclosure choose a model for [symbol] (y(0),y(1)|X=x; Θ_{Y}) that can incorporate population-level information in the form of the ATE. In some examples, the chosen model is based on Gaussian processes because, for example, they automatically enable uncertainty quantification. In other examples, other models of choice can be used.

In matrix form, the dataset is expressed as [symbol] ={(y₀, X₀), (y₁, X₁)} containing n=n₀+n₁ data points, where n₀ corresponds to the control group and n₁ corresponds to the treatment group, y=[y₀, y₁], and X=[X₀, X₁]. In functional form, the potential outcomes are expressed as:

$$Y ( A = a ) = {f}_{a} ( x ) + ϵ , for a = 0 , 1$$

where ϵ˜ [symbol] (0, σ²) (e.g., same noise model for both outcomes). In the example, ƒ=[ƒ₀, ƒ₁] is modelled as a vector-values Gaussian process with zero mean and intrinsic coregionalization K_{a,a′}(x, x′)=CoV(ƒₐ(X),ƒ_{a′}(x′)):=B_{a,a′}K(x, x′). B ∈ [symbol] that simultaneously captures the correlation between inputs (covariates) and outcomes (and serves to impose assumptions on ƒ₀ and ƒ₁). K: X×X→ [symbol] is positive definite covariance operator.

Further, it is denoted by ƒₐ, a=0.1 the vector such that (ƒₐ)ᵢ=ƒ((Xₐ)ᵢ). Therefore, ƒ₀ and ƒ₁ follows a Gaussian distribution:

$$( \begin{matrix}{f}_{0} \\ {f}_{1}\end{matrix} ) ∼ GP ( [ \begin{matrix}0 \\ 0\end{matrix} ] , K = [ \begin{matrix}{K}_{00} & {K}_{01} \\ {K}_{10} & {K}_{11}\end{matrix} ] ) ,$$

where K_{a,a′} is a covariance matrix such that (K_{a,a′})ᵢⱼ=K_{a,a′}(x_{a′i}, x_{a′j}). The log-marginal likelihood of this model is the result of integrating out ƒ:

$$\begin{matrix}log ℙ ( y \vert X ; Θ ) = \int ℙ ( y \vert f , X ) ℙ ( f \vert X ) df \\ = log p ( y \vert X ) \\ = - \frac{1}{2} {y}^{T} {( K + {σ}^{2} I )}^{- 1} y - \frac{1}{2} log \vert K + {σ}^{2} I \vert - \frac{n}{2} log 2 π ,\end{matrix}$$

where Θ includes the parameters of the kernel K, B, and σ². These parameters can be optimized by gradient descent to fit the model to the two potential outcomes.

An induced probability measure on the ATE, given the model that establishes a probability measure on the vector-valued function ƒ, is shown below:

$${τ}_{f} = {\int }_{X} ( {f}_{1} ( x ) - {f}_{0} ( x ) ) ℙ ( x ) d x = {\int }_{X} ( {f}_{1} ( x ) ) ℙ ( x ) d x - {\int }_{X} ( {f}_{0} ( x ) ) ℙ ( x ) dx = {I}_{1} - {I}_{0} .$$

This measure is a probabilistic estimand of the ATE, assuming the model of ƒ is rightly specified. Because of the properties of Gaussians, this integral process is also Gaussian, since it is the result of linear transformations (e.g., integral and subtraction) of Gaussian distributed variables. It is possible to show that Iₐ˜ [symbol] (0, v_{I}_{ₐ}), where:

$${v}_{{I}_{a}} = {\int }_{X} {K}_{a , a} ( x , {x}^{′} ) ℙ ( x ) d x ℙ ( {x}^{′} ) d {x}^{′} ,$$

for a=0, 1. The covariance between the integrals I₁ and I₀ is:

$${c}_{{I}_{{aa}^{′}}} = {\int }_{X} {K}_{a , {a}^{′}} ( {x}_{x} {x}^{′} ) ℙ ( x ) d x ℙ ( {x}^{′} ) d {x}^{′} ,$$

and using the properties the covariance, the induced probability measure on τ_{ƒ} given a GP 122 on ƒ with the covariance detailed above is:

$${τ}_{f} ∼ 𝒩 ( 0 , {k}_{τ , τ} = {v}_{{I}_{0}} + {v}_{{I}_{1}} + {c}_{{I}_{a , {a}^{′}}} ) .$$

Next, this result is used to define a prior over ƒ that incorporates previous information about the ATE. More specifically, a prior is derived over ƒ constrained to if taking certain predefined value T. To achieve this, a joint measure over (ƒ₀, ƒ₁, τ_{ƒ}) is derived. Then a conditional is computed as (ƒ₀, ƒ1)|Tf=T. This corresponds to a Gaussian process with a specific form of prior mean and covariance.

(ƒ₀, ƒ₁, τ_{ƒ}) has mean zero by construction. From the covariance structure of (ƒ₀, ƒ₁, τ_{ƒ}), the only block that has not yet been derived is the one corresponding to the Cov(ƒₐ(x′), τ_{ƒ}). Applying the properties of the covariance yields:

$$\begin{matrix}Cov ( {f}_{a} ( {x}^{′} ) , {τ}_{f} ) = Cov ( {f}_{a} ( {x}^{′} ) , {I}_{1} - {I}_{0} ) \\ = Cov ( {f}_{a} ( {x}^{′} ) , {I}_{1} ) - Cov ( {f}_{a} ( {x}^{′} ) , {I}_{0} ) ) \\ = {\int }_{X} {K}_{a , 1} ( {x}^{′} , x ) dx - {\int }_{X} {K}_{a , 0} ( {x}^{′} , x ) dx .\end{matrix}$$

For a given input matrix Xₐ, the vector is defined as:

$${z}_{{aa}^{′}} ( {X}_{a} ) = {[ \int {K}_{{aa}^{′}} ( {x}_{1} , x ) ℙ ( x ) dx , … , {K}_{{aa}^{′}} ( {x}_{m} , x ) ℙ ( x ) d x ]}^{T} , ( \begin{matrix}{f}_{0} \\ {f}_{1} \\ {τ}_{f}\end{matrix} ) ∼ GP ( [ \begin{matrix}0 \\ 0 \\ 0\end{matrix} ] , \text{} C = ( \begin{matrix}{K}_{00} & {K}_{01} & {z}_{01} ( {X}_{0} ) - {z}_{00} ( {X}_{0} ) \\ {K}_{10} & {K}_{11} & {z}_{11} ( {X}_{1} ) - {z}_{10} ( {X}_{1} ) \\ {z}_{01} ( {X}_{0} ) - {z}_{00} ( {X}_{0} ) & {z}_{11} ( {X}_{1} ) - {z}_{10} ( {X}_{1} ) & {k}_{r , r}\end{matrix} ) ) .$$

Next, the mean and variance of ƒ₀, ƒ₁|(τ_{ƒ}=t) is computed. To simplify notation, ƒ₀, ƒ₁ is expressed simply as ƒ, which results in:

$$( \begin{matrix}f \\ {τ}_{f}\end{matrix} ) ∼ GP ( [ \begin{matrix}0 \\ 0\end{matrix} ] , C = [ \begin{matrix}K & {k}_{f , τ} \\ {k}_{τ , f} & {k}_{τ , τ}\end{matrix} ] ) ,$$

where the correspondence with the representation above is straightforward by taking:

$${k}_{τ , f} = [ {z}_{01} ( {X}_{0} ) - {z}_{00} ( {X}_{0} ) , {z}_{11} ( {X}_{1} ) - {z}_{10} ( {X}_{1} ) ] .$$

Finally obtained is that:

$$f \vert ( {τ}_{f} = t ) ∼ 𝒩 ( μ = \frac{t}{{k}_{τ , τ}} {k}_{f , τ} , \sum = K - \frac{1}{{k}_{τ , τ}} {k}_{f , τ} {k}_{τ , f} ) .$$

The estimation of the CATE, for a given individual, and using information about the known ATE=t, is the difference between the outputs of the GP 122 that uses a prior with the above mean and covariance.

As such, the GP 122 is trained for ƒ with the above prior mean and variance. The GP 122 thus integrates known data about a characteristic of a population (e.g., the population ATE 126) to infer something about individuals (e.g., the difference between ƒ₀ and ƒ₁, how a patient having that characteristic would respond to a particular treatment).

Referring again to FIG. 1, the interface engine 130 is configured to use the GP model 122 during a production phase. At this stage, the PH system 100 deploys the GP model 122 for clinical use, allowing clinicians 152 to interface with the PH device 110 and use the GP model 122 to generate CATE estimations for particular patients 154.

In examples, the clinician 152 uses a client computing device 156 to interact with the PH device 110 and associated functions (e.g., via an application programming interface (API), or the like). For example, the interface engine 130 allows the clinician 152 to identify the particular patient 154 and trigger a CATE estimation for that patient and a particular medical treatment of interest (e.g., the treatment upon which the GP model 122 was trained). The interface engine 130 identifies patient data 132 for the particular patient 154 (e.g., accessed securely and anonymously from a patient data DB 116, or the like) and uses features of this patient data 132 as input to the GP model 122. The GP model 122 generates GP output 134, which includes the CATE estimation for the particular patient 154 given the particular treatment.

In some examples, many GP models 122 may be trained and deployed for use by the PH device 110. For example, different GP models 122 may be trained for different treatments, or for the same treatment using different populations (e.g., different observational data 124, different population ATE 126).

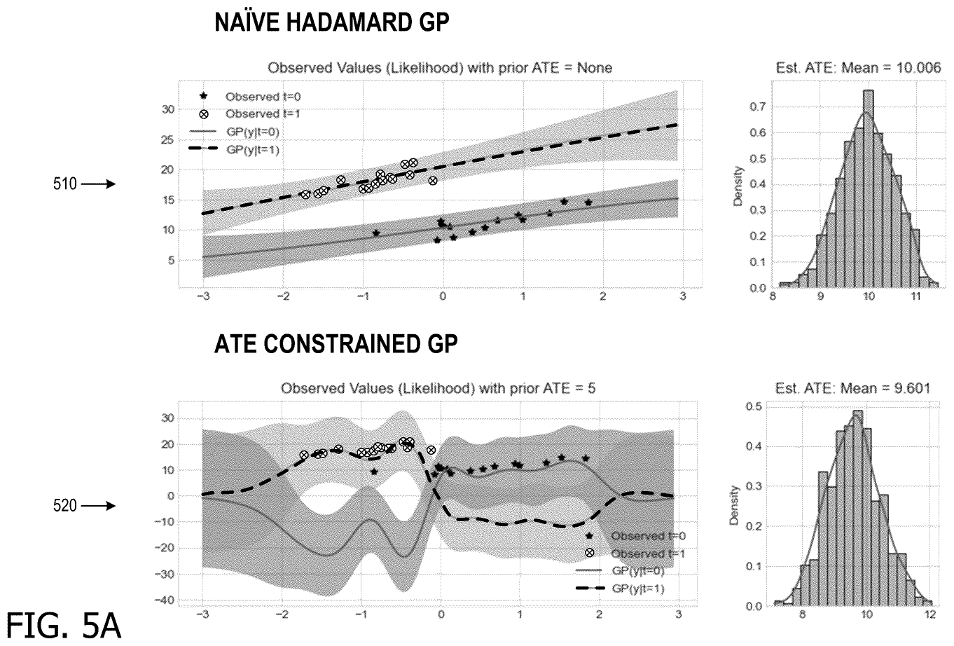

FIG. 4 is a graph 400 plotting example observational data 124 that may be used to train the GP model 122 of FIG. 1. In the example, the X-axis 402 represents a transformed patient age, and a Y-axis 404 represents some measurable outcome value (e.g., a number of days in recovery). The plot shown in the example graph 400 displays a treated population in X data points and an untreated population in solid data points, where each data point is from the observational data 124 (e.g., based on an example drug treatment). It should be noted that the treated population, in this example, tends to be younger, and the untreated population tends to be older. In this example, when age is controlled for, the ATE is approximately 4.5. However, if the GP model 122 were trained naïvely (e.g., without consideration for the ATE), estimates of treatment effect for a given patient are less accurate.

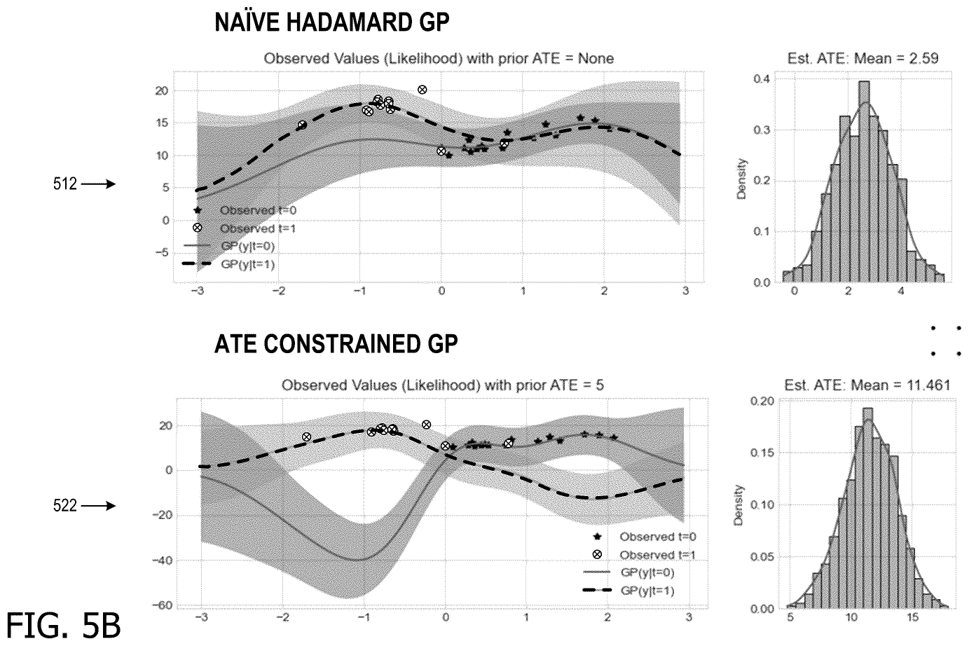

FIG. 5A and FIG. 5B illustrate differences between an example naïve approach to training a GP for CATE estimations versus the example GP training as described herein. In the example, graphs 510, 512 illustrate an example naïve approach in which a Hadamard Gaussian process is used to train a GP model (e.g., without consideration of ATE), and graphs 520, 522 illustrate the GP training process described herein (e.g., using a prior ATE=5). Each of the graphs 510-522 plot observed values (t=0) and GP(y|t=0) for untreated patients (solid line), as well as observed values (t=1) and GP(y|t=1) for treated patients (dashed line). The graphs 510, 520 of FIG. 5A represent one example treatment and the results of some particular trained GPs (e.g., naïve approach and novel approach, respectively), and the graphs 512, 522 of FIG. 5B represent another example treatment and results of other trained GPs (e.g., naïve approach and novel approach, respectively).

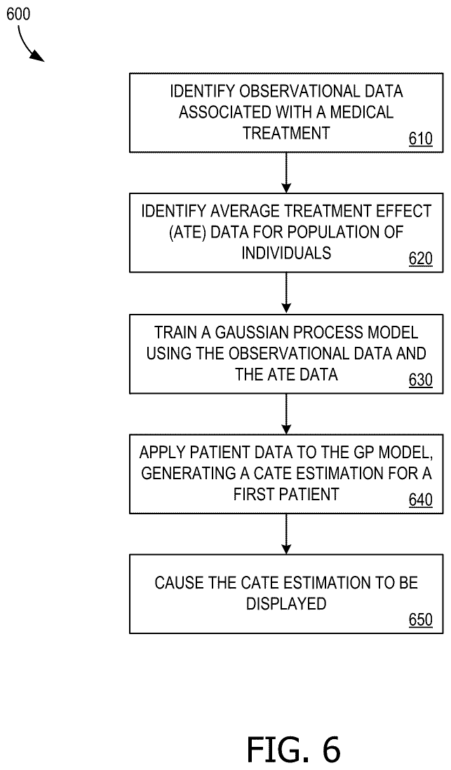

FIG. 6 is a flowchart 600 illustrating exemplary operations that may be performed by the PH system 100 of FIG. 1 for providing a GP-based framework that incorporates population-level information into an estimation procedure of a CATE. In some examples, the operations of flowchart 600 may be similar to the operations shown and described in FIG. 1. In the example implementation, the operations of flowchart 600 are performed by the PH device 110 of FIG. 1.

At operation 610, the PH device 110 identifies observational data (e.g., observational data 124) associated with a medical treatment (e.g., a drug therapy, drug regimen, or the like). In some examples, the observational data includes treatment effect data associated with a plurality of control individuals not receiving the medical treatment and a plurality of treated individuals having received the medical treatment. At operation 620, the PH device 110 identifies ATE data (e.g., population ATE data 126) associated with the medical treatment performed across a population of individuals.

At operation 630, the PH device 110 trains a GP model (e.g., GP model 122) using at least the observational data 124 and the ATE data 126, the GP model 122 being trained to generate at least a conditional average treatment effect (CATE) estimation (e.g., as GP output 134) for the medical treatment. In some examples, training the GP model 122 includes determining a first function (e.g., ƒ₀) and a second function (e.g., ƒ₁), the first function being related to untreated subject estimations for the medical treatment, the second function being related to treated subject estimations for the medical treatment. In some examples, training the GP model 122 further uses a mean function and a covariance function, the mean function and the covariance function being identified based on user input via an administrative interface (e.g., UI 106).

At operation 640, the PH device 110 applies patient data (e.g., patient data 132) of a first patient (e.g., patient 154) as input to the GP model 122, thereby generating a first CATE estimation identifying an estimation of how the medical treatment would affect the first patient 154. In some examples, generating the first CATE estimation includes determining a difference between an untreated subject estimation function and a treated subject estimation function. At operation 650, the PH device 110 causes the first CATE estimation to be displayed to a clinician (e.g., clinician 152) during consideration of applying the medical treatment to the first patient 154. In other examples. The PH device 110 causes treatment to be applied to the first patient 154, such as by automatically administering medication, instructing the clinician 152 to administer treatment, and/or the like.

In some examples, the PH device 110 causes a first graph (e.g., graph 520, 522) to be displayed to the clinician 152, the first graph 520, 522 including at least a representation of a first function generated using the GP model 122 and related to untreated subject estimations and a second function generated using the GP model 122 and related to treated subject estimations. In some examples, the PH device 110 trains a plurality of GP models 122, each GP model 122 being trained with different observational data 124 and different ATE data 126 regarding a different medical treatment, and generates one or more additional CATE estimations identifying an estimation of one or more other medical treatments would affect the first patient 154.

### Additional Examples

An example precision health system comprises: a processor executing instructions that cause the processor to: identify observational data associated with a medical treatment; identify ATE data associated with the medical treatment performed across a population of individuals; train a GP model using at least the observational data and the ATE data, the GP model being trained to generate at least a CATE estimation for the medical treatment; apply patient data of a first patient as input to the GP model, thereby generating a first CATE estimation identifying an estimation of how the medical treatment would affect the first patient; and cause the first CATE estimation to be displayed to a clinician during consideration of applying the medical treatment to the first patient.

An example computer-implemented method comprises: receiving observational data associated with a medical treatment; receiving ATE data associated with the medical treatment performed across a population of individuals; training a GP model using at least the observational data and the ATE data, the GP model being trained to generate at least a CATE estimation for the medical treatment; applying patient data of a first patient as input to the GP model, thereby generating a first CATE estimation identifying an estimation of how the medical treatment would affect the first patient; and causing the first CATE estimation to be displayed.

An example computer storage device has computer-executable instructions stored thereon, which, on execution by a computer, cause the computer to perform operations comprising: receiving observational data associated with a medical treatment; receiving ATE data associated with the medical treatment performed across a population of individuals; training a GP model using at least the observational data and the ATE data, the GP model being trained to generate at least a CATE estimation for the medical treatment; applying patient data of a first patient as input to the GP model, thereby generating a first CATE estimation identifying an estimation of how the medical treatment would affect the first patient; and causing the first CATE estimation to be displayed.

Alternatively, or in addition to the other examples described herein, examples include any combination of the following:
receiving observational data associated with a medical treatment;
receiving average treatment effect (ATE) data associated with the medical treatment performed across a population of individuals;
training a Gaussian process (GP) model using at least the observational data and the ATE data, the GP model being trained to generate at least a conditional average treatment effect (CATE) estimation for the medical treatment;
training a Gaussian process model;
training a GP model using at least observational data;
training a GP model using at least ATE data;
applying patient data of a first patient as input to the GP model, thereby generating a first CATE estimation identifying an estimation of how the medical treatment would affect the first patient;
causing CATE estimations to be displayed;
displaying CATE estimations to a clinician;
receiving a request from a clinician computing device to apply patient data to a GP model;
causing a first graph to be displayed to the clinician;
a graph including at least a representation of a first function generated using the GP model and related to untreated subject estimations;
a graph including at least a second function generated using the GP model and related to treated subject estimations;
observational data that includes treatment effect data associated with a plurality of control individuals not receiving the medical treatment and a plurality of treated individuals having received the medical treatment;
training a GP model includes determining a first function and a second function, the first function being related to untreated subject estimations for the medical treatment, the second function being related to treated subject estimations for the medical treatment;
training the GP model further uses a mean function and a covariance function, the mean function and covariance function being identified based on user input via an administrative interface;
generating the first CATE estimation includes determining a difference between an untreated subject estimation function and a treated subject estimation function;
training a plurality of GP models, each GP model being trained with different observational data and different ATE data regarding a different medical treatment;
and
generating one or more additional CATE estimations identifying an estimation of one or more other medical treatments would affect the first patient.

While the aspects of the disclosure have been described in terms of various examples with their associated operations, a person skilled in the art would appreciate that a combination of operations from any number of different examples is also within scope of the aspects of the disclosure.

### Example Operating Environment

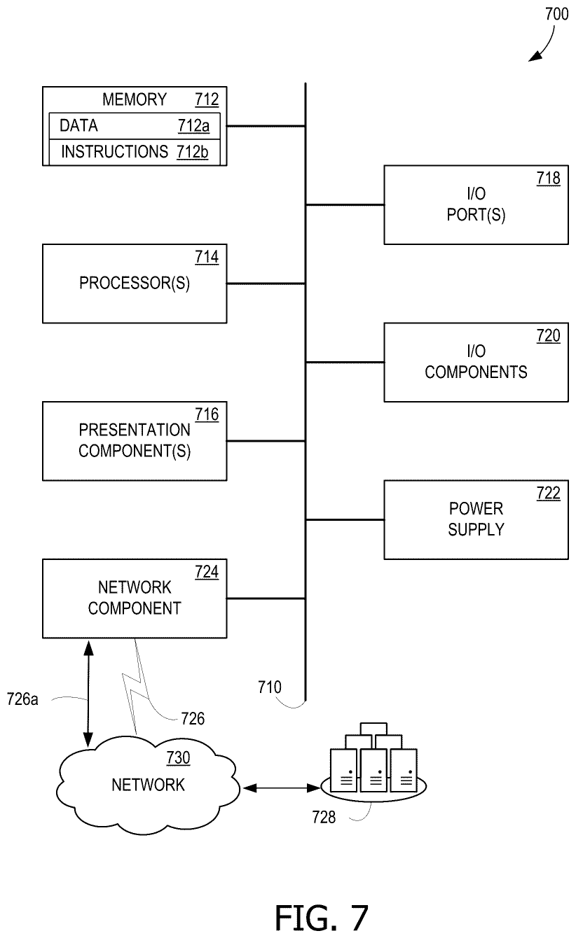

FIG. 7 is a block diagram of an example computing device 700 for implementing aspects disclosed herein and is designated generally as computing device 700. In some examples, computing device 104, 156, and PH device 110 may be similar to computing device 700. In some examples, one or more computing devices 700 are provided for an on-premises computing solution. In some examples, one or more computing devices 700 are provided as a cloud computing solution. In some examples, a combination of on-premises and cloud computing solutions are used. Computing device 700 is but one example of a suitable computing environment and is not intended to suggest any limitation as to the scope of use or functionality of the examples disclosed herein, whether used singly or as part of a larger set. Neither should computing device 700 be interpreted as having any dependency or requirement relating to any one or combination of components/modules illustrated.

The examples disclosed herein may be described in the general context of computer code or machine-useable instructions, including computer-executable instructions such as program components, being executed by a computer or other machine, such as a personal data assistant or other handheld device. Generally, program components including routines, programs, objects, components, data structures, and the like, refer to code that performs particular tasks, or implement particular abstract data types. The disclosed examples may be practiced in a variety of system configurations, including personal computers, laptops, smart phones, mobile tablets, hand-held devices, consumer electronics, specialty computing devices, etc. The disclosed examples may also be practiced in distributed computing environments when tasks are performed by remote-processing devices that are linked through a communications network.

Computing device 700 includes a bus 710 that directly or indirectly couples the following devices: computer storage memory 712, one or more processors 714, one or more presentation components 716, input/output (I/O) ports 718, I/O components 720, a power supply 722, and a network component 724. While computing device 700 is depicted as a seemingly single device, multiple computing devices 700 may work together and share the depicted device resources. For example, memory 712 may be distributed across multiple devices, and processor(s) 714 may be housed with different devices.

Bus 710 represents what may be one or more busses (such as an address bus, data bus, or a combination thereof). Although the various blocks of FIG. 6 are shown with lines for the sake of clarity, delineating various components may be accomplished with alternative representations. For example, a presentation component such as a display device is an I/O component in some examples, and some examples of processors have their own memory. Distinction is not made between such categories as “workstation,” “server,” “laptop,” “hand-held device,” etc., as all are contemplated within the scope of FIG. 6 and the references herein to a “computing device.” Memory 712 may take the form of the computer storage media referenced below and operatively provide storage of computer-readable instructions, data structures, program modules and other data for the computing device 700. In some examples, memory 712 stores one or more of an operating system, a universal application platform, or other program modules and program data. Memory 712 is thus able to store and access data 712a and instructions 712b that are executable by processor 714 and configured to carry out the various operations disclosed herein.

In some examples, memory 712 includes computer storage media. Memory 712 may include any quantity of memory associated with or accessible by the computing device 700. Memory 712 may be internal to the computing device 700 (as shown in FIG. 6), external to the computing device 700 (not shown), or both (not shown). Additionally, or alternatively, the memory 712 may be distributed across multiple computing devices 700, for example, in a virtualized environment in which instruction processing is carried out on multiple computing devices 700. For the purposes of this disclosure, “computer storage media,” “computer-storage memory,” “memory,” and “memory devices” are synonymous terms for the computer-storage memory 712, and none of these terms include carrier waves or propagating signaling.

Processor(s) 714 may include any quantity of processing units that read data from various entities, such as memory 712 or I/O components 720. Specifically, processor(s) 714 are programmed to execute computer-executable instructions for implementing aspects of the disclosure. The instructions may be performed by the processor, by multiple processors within the computing device 700, or by a processor external to the client computing device 700. In some examples, the processor(s) 714 are programmed to execute instructions such as those illustrated in the flow charts discussed below and depicted in the accompanying drawings. Moreover, in some examples, the processor(s) 714 represent an implementation of analog techniques to perform the operations described herein. For example, the operations may be performed by an analog client computing device 700 and/or a digital client computing device 700. Presentation component(s) 716 present data indications to a user or other device. Exemplary presentation components include a display device, speaker, printing component, vibrating component, etc. One skilled in the art will understand and appreciate that computer data may be presented in a number of ways, such as visually in a graphical user interface (GUI), audibly through speakers, wirelessly between computing devices 700, across a wired connection, or in other ways. I/O ports 718 allow computing device 700 to be logically coupled to other devices including I/O components 720, some of which may be built in. Example I/O components 720 include, for example but without limitation, a microphone, joystick, game pad, satellite dish, scanner, printer, wireless device, etc.

Computing device 700 may operate in a networked environment via the network component 724 using logical connections to one or more remote computers. In some examples, the network component 724 includes a network interface card and/or computer-executable instructions (e.g., a driver) for operating the network interface card. Communication between the computing device 700 and other devices may occur using any protocol or mechanism over any wired or wireless connection. In some examples, network component 724 is operable to communicate data over public, private, or hybrid (public and private) using a transfer protocol, between devices wirelessly using short range communication technologies (e.g., near-field communication (NFC), Bluetooth™ branded communications, or the like), or a combination thereof. Network component 724 communicates over wireless communication link 726 and/or a wired communication link 726a to a remote resource 728 (e.g., a cloud resource) across network 730. Various different examples of communication links 726 and 726a include a wireless connection, a wired connection, and/or a dedicated link, and in some examples, at least a portion is routed through the internet.

Although described in connection with an example computing device 700, examples of the disclosure are capable of implementation with numerous other general-purpose or special-purpose computing system environments, configurations, or devices. Examples of well-known computing systems, environments, and/or configurations that may be suitable for use with aspects of the disclosure include, but are not limited to, smart phones, mobile tablets, mobile computing devices, personal computers, server computers, hand-held or laptop devices, multiprocessor systems, gaming consoles, microprocessor-based systems, set top boxes, programmable consumer electronics, mobile telephones, mobile computing and/or communication devices in wearable or accessory form factors (e.g., watches, glasses, headsets, or earphones), network PCs, minicomputers, mainframe computers, distributed computing environments that include any of the above systems or devices, virtual reality (VR) devices, augmented reality (AR) devices, mixed reality devices, holographic device, and the like. Such systems or devices may accept input from the user in any way, including from input devices such as a keyboard or pointing device, via gesture input, proximity input (such as by hovering), and/or via voice input.

Examples of the disclosure may be described in the general context of computer-executable instructions, such as program modules, executed by one or more computers or other devices in software, firmware, hardware, or a combination thereof. The computer-executable instructions may be organized into one or more computer-executable components or modules. Generally, program modules include, but are not limited to, routines, programs, objects, components, and data structures that perform particular tasks or implement particular abstract data types. Aspects of the disclosure may be implemented with any number and organization of such components or modules. For example, aspects of the disclosure are not limited to the specific computer-executable instructions or the specific components or modules illustrated in the figures and described herein. Other examples of the disclosure may include different computer-executable instructions or components having more or less functionality than illustrated and described herein. In examples involving a general-purpose computer, aspects of the disclosure transform the general-purpose computer into a special-purpose computing device when configured to execute the instructions described herein.

By way of example and not limitation, computer readable media comprise computer storage media and communication media. Computer storage media include volatile and nonvolatile, removable and non-removable memory implemented in any method or technology for storage of information such as computer readable instructions, data structures, program modules, or the like. Computer storage media are tangible and mutually exclusive to communication media. Computer storage media are implemented in hardware and exclude carrier waves and propagated signals. Computer storage media for purposes of this disclosure are not signals. Exemplary computer storage media include hard disks, flash drives, solid-state memory, phase change random-access memory (PRAM), static random-access memory (SRAM), dynamic random-access memory (DRAM), other types of random-access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transmission medium that may be used to store information for access by a computing device. In contrast, communication media typically embody computer readable instructions, data structures, program modules, or the like in a modulated data signal such as a carrier wave or other transport mechanism and include any information delivery media.

The order of execution or performance of the operations in examples of the disclosure illustrated and described herein is not essential, and may be performed in different sequential manners in various examples. For example, it is contemplated that executing or performing a particular operation before, contemporaneously with, or after another operation is within the scope of aspects of the disclosure. When introducing elements of aspects of the disclosure or the examples thereof, the articles “a,” “an,” “the,” and “said” are intended to mean that there are one or more of the elements. The terms “comprising,” “including,” and “having” are intended to be inclusive and mean that there may be additional elements other than the listed elements. The term “exemplary” is intended to mean “an example of.” The phrase “one or more of the following: A, B, and C” means “at least one of A and/or at least one of B and/or at least one of C.”

Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

What is claimed is:

1. A precision health device comprising:
a processor executing instructions that cause the processor to:
identify observational data associated with a medical treatment;
identify average treatment effect (ATE) data associated with the medical treatment performed across a population of individuals;
train a Gaussian process (GP) model using at least the observational data and the ATE data, the GP model being trained to generate at least a conditional average treatment effect (CATE) estimation for the medical treatment;
apply patient data of a first patient as input to the GP model, thereby generating a first CATE estimation identifying an estimation of how the medical treatment would affect the first patient; and
cause the medical treatment to be applied to the first patient based on the first CATE estimation.

2. The precision health device of claim 1, wherein the instructions further cause the processor to cause a first graph to be displayed to a clinician, the first graph including at least a representation of a first function generated using the GP model and related to untreated subject estimations and a second function generated using the GP model and related to treated subject estimations.

3. The precision health device of claim 1, wherein the observational data includes treatment effect data associated with a plurality of control individuals not receiving the medical treatment and a plurality of treated individuals having received the medical treatment.

4. The precision health device of claim 1, wherein training the GP model includes determining a first function and a second function, the first function being related to untreated subject estimations for the medical treatment, the second function being related to treated subject estimations for the medical treatment.

5. The precision health device of claim 1, wherein training the GP model further uses a mean function and a covariance function, the mean function and the covariance function being identified based on user input via an administrative interface.

6. The precision health device of claim 1, wherein generating the first CATE estimation includes determining a difference between an untreated subject estimation function and a treated subject estimation function.

7. The precision health device of claim 1, wherein the instructions further cause the processor to:
train a plurality of GP models, each GP model being trained with different observational data and different ATE data regarding a different medical treatment; and
generate one or more additional CATE estimations identifying an estimation of how one or more other medical treatments would affect the first patient.

8. A computer-implemented method comprising:
receiving observational data associated with a medical treatment;
receiving average treatment effect (ATE) data associated with the medical treatment performed across a population of individuals;
training a model using at least the observational data and the ATE data, the model being trained to generate at least a conditional average treatment effect (CATE) estimation for the medical treatment;
applying patient data of a first patient as input to the model, thereby generating a first CATE estimation identifying an estimation of how the medical treatment would affect the first patient; and
causing the first CATE estimation to be displayed.

9. The method of claim 8, further comprising causing a first graph to be displayed to a clinician, the first graph including at least a representation of a first function generated using the model and related to untreated subject estimations and a second function generated using the model and related to treated subject estimations.

10. The method of claim 8, wherein the observational data includes treatment effect data associated with a plurality of control individuals not receiving the medical treatment and a plurality of treated individuals having received the medical treatment.

11. The method of claim 8, wherein training the model includes determining a first function and a second function, the first function being related to untreated subject estimations for the medical treatment, the second function being related to treated subject estimations for the medical treatment.

12. The method of claim 8, wherein training the model further uses a mean function and a covariance function, the mean function and the covariance function being identified based on user input via an administrative interface.

13. The method of claim 8, wherein generating the first CATE estimation includes determining a difference between an untreated subject estimation function and a treated subject estimation function.

14. The method of claim 8, further comprising:
training a plurality of models, each of the plurality of models being trained with different observational data and different ATE data regarding a different medical treatment; and
generating one or more additional CATE estimations identifying an estimation of one or more other medical treatments would affect the first patient.

15. A computer storage device having computer-executable instructions stored thereon, which, on execution by a computer, cause the computer to perform operations comprising:
receiving observational data associated with a medical treatment;
receiving average treatment effect (ATE) data associated with the medical treatment performed across a population of individuals;
training a Gaussian process (GP) model using at least the observational data and the ATE data, the GP model being trained to generate at least a conditional average treatment effect (CATE) estimation for the medical treatment;
applying patient data of a first patient as input to the GP model, thereby generating a first CATE estimation identifying an estimation of how the medical treatment would affect the first patient; and
causing the first CATE estimation to be displayed to a clinician during consideration of applying the medical treatment to the first patient.

16. The computer storage device of claim 15, the operations further comprising causing a first graph to be displayed to the clinician, the first graph including at least a representation of a first function generated using the GP model and related to untreated subject estimations and a second function generated using the GP model and related to treated subject estimations.

17. The computer storage device of claim 15, wherein the observational data includes treatment effect data associated with a plurality of control individuals not receiving the medical treatment and a plurality of treated individuals having received the medical treatment.

18. The computer storage device of claim 15, wherein training GP model includes determining a first function and a second function, the first function being related to untreated subject estimations for the medical treatment, the second function being related to treated subject estimations for the medical treatment.

19. The computer storage device of claim 15, wherein training the GP model further uses a mean function and a covariance function, the mean function and the covariance function being identified based on user input via an administrative interface, wherein generating the first CATE estimation includes determining a difference between an untreated subject estimation function and a treated subject estimation function.

20. The computer storage device of claim 15, the operations further comprising:
training a plurality of GP models, each GP model being trained with different observational data and different ATE data regarding a different medical treatment; and
generating one or more additional CATE estimations identifying an estimation of one or more other medical treatments would affect the first patient.
